(19) United States
(12) Patent Application Publication
(10) Pub. No.: US 2002/0122782 A1
(43) Pub. Date: Sep. 5, 2002

(54) Hair treatment system

(76) Inventor: Gwen-Nelson Bichard, Putnam, CT (US)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/102,466

(22) Filed: Mar. 20, 2002

Related U.S. Application Data
(63) Continuation-in-part of application No. 09/688,418, filed on Oct. 16, 2000, now abandoned.
(60) Provisional application No. 60/159,911, filed on Oct. 15, 1999.

(51) Int. Cl. A61K 7/11; A61K 7/06

(52) U.S. Cl. 424/70.11

(57) ABSTRACT

The present invention provides for a safe non-toxic hair treatment composition, a hair treatment system and a method of using such hair treatment system. The hair treatment composition preferably comprises a non-toxic adhesive polymer, such as a polymer of methyl vinyl ether/maleic acid and an antioxidant.

The present application is a continuation-in-part of U.S. patent application Ser. No. 09/688,418, filed on Oct. 16, 2000, which claims the benefit of provisional application no. 60/159,911 filed Oct. 15, 1999, the contents of which are hereby incorporated by reference.

### FIELD OF THE INVENTION

The present invention relates to hair treatment formulations and methods of use providing for safer, less-toxic, hair extension treatments. The hair treatment formulations and methods include a non-toxic polymer selected for its adhesive properties.

### BACKGROUND OF THE INVENTION

Hair extension is a process whereby a new strand of natural or synthetic hair (the extension strand) of any desired length is attached to a customer's existing hair or scalp thereby extending the perceived natural length, fullness, shape or style of a customer's hair. There are two common methods currently used in the hair extension process, both of these methods are typically referred to as “weaving.”

The first weaving method is accomplished by systematically braiding the customer's natural hair so as to create a framework for attaching the extension strands. The extension strands are then actually sewn into the braids of the customer's natural hair. This method is extremely time consuming, often taking 4-5 hours to complete. This is inconvenient; especially considering that typical “weaves” last only a couple of weeks at best.

To shorten the time it takes for the hair stylist to complete a “weave,” a second method utilizing harsh chemicals such as rubber latex or isoprene butadiene is used. This method starts with extension strands, which are tied or otherwise attached to a string, or other support. Next, the hair on the customer's head is parted, typically near the crown of the head, thereby exposing a portion of the customer's scalp. The extension strands are then bonded to the scalp at the string by applying an amount of the rubber latex or isoprene butadiene and allowing it to dry. The process is repeated by creating another part in the customer's hair next to the previous part and working away from the crown of the head (typically in both directions) until all the desired area of the customer's head is treated. This method is very effective and much faster, typically taking about 1½ to 2 hours for a hair stylist to complete.

Unfortunately, this method also has major drawbacks. First, the rubber latex or isoprene butadiene is extremely irritating to the skin and the mucous membranes of the customer. Those customers who continuously visit hair stylists for hair extensions constantly have very sore, raw, irritated scalps. It is not uncommon that these raw scalps develop scabs, which in turn at times bleed.

Second, the hair stylists who practice this method must apply the materials with gloves, aprons, surgical masks and the like. Indeed, hair stylists work with these compositions on a daily basis, greatly increasing their exposure to potential health risks due to long-term exposure to these irritating compositions.

Lastly, there is considerable talk in the industry that the treatment of hair with these harsh irritating compositions may cause significant damage to the customer's hair, including permanent hair loss.

These problems are further exacerbated when considering the treatment of especially difficult to treat hair such as that of the African-American community. Treating especially difficult hair typically requires even harsher chemicals increasing the potential irritation and health risks to both customers and hair stylists alike.

Accordingly, it would be desirable to provide hair stylists and customers with a non-toxic hair treatment composition that is as effective as rubber latex or isoprene butadiene while simultaneously eliminating the irritating properties and health risks associated with these known compositions in current use. There are known non-toxic polymers which are utilized in hair formulations such as hair sprays, shampoos and the like for their film forming properties. However, these non-toxic polymers have been used in significantly small concentrations and not for their adhesive properties. They were also not used in products intended for long-term exposure to a customer's scalp. Therefore, the behavior, performance, and low irritancy under such conditions were unknown. Most importantly in the context of the present invention these non-toxic polymers have not been used to bond an extension strand to the customer's scalp.

There are also other products which utilize adhesive polymers such as methyl vinyl ether/maleic anhydride for their adhesive properties in applications such as repairing horse hooves and as dental adhesives. However, these polymers are not non-toxic and to date have not been used as an adhesive for hair.

Therefore, there is a need in the art for a safe, effective, non-toxic adhesive polymer for use in hair extension processes for hair-hair and hair-scalp bonding. To date, there has not been a hair treatment composition that satisfactorily meets this need.

### SUMMARY OF THE INVENTION

The present invention solves this long felt need by providing a hair treatment composition capable of providing the necessary performance advantages of an adhesive such as rubber latex or isoprene butadiene without the irritation and potential long-term health risks associated with use of these chemicals. It has been unexpectedly found that by selecting the current class of adhesive compositions, one can obtain the performance of prior adhesive bonding methods without toxicity and with reduced irritation.

According to one aspect of the present invention, a hair treatment composition is provided for bonding extension strands to a human scalp or to hair proximate to a human scalp comprising about 50% to about 95% by weight of a non-toxic adhesive polymer, with the balance being a mixture of water and antioxidants.

According to another aspect of the present invention, a hair extension and removal system is provided comprising a hair treatment composition and a hair bond remover. The hair bond remover comprises an amount of the same non-toxic adhesive polymer as the hair treatment composition sufficient to loosen a bond created by applying an amount of the hair treatment composition and allow for removal of an extension strand from a customer's scalp or customer's hair.

According to another aspect of the present invention, a method of extending the length of a customer's hair is provided comprising the steps of providing a customer who desires to increase the length of his/her hair, parting the hair on the customer's head thereby exposing a portion of the customer's scalp or customer's hair proximate to the scalp, placing an extension strand proximate to the scalp or to hair proximate to the scalp, applying an effective amount of a hair treatment composition having about 50% to about 95% by weight of a non-toxic adhesive polymer with the balance being a mixture of antioxidants and water to an area of the scalp or to hair proximate to the scalp which is sufficient to bond the extension strand to the scalp or to hair proximate to the scalp and allowing the hair treatment composition to dry thereby bonding the extension strand to the scalp to hair proximate to the scalp. Preferably, the non-toxic adhesive polymer is selected from the group consisting of a polymer of methyl vinyl ether/maleic acid, a butyl ester of said polymer of methyl vinyl ether/maleic acid, an ethyl ester of said polymer of methyl vinyl ether/maleic acid, and an isopropyl ester of said polymer of methyl vinyl ether/maleic acid.

According to yet another aspect of the present invention, a method for removing a hair extension from a customer's scalp or from hair proximate to the customer's scalp is provided comprising the steps of applying an amount of hair bond remover having an amount of non-toxic adhesive sufficient to loosen or re-liquefy a bond between an extension strand and a customer's scalp or customer's hair proximate to the scalp that was created using a hair treatment composition having an amount of non-toxic adhesive polymer and separating the extension strand from the customer's scalp or the customer's hair proximate to the scalp.

### DETAILED DESCRIPTION OF PREFERRED EMBODIMENTS

As previously discussed, the present invention relates to the safe, non-toxic treatment of hair utilizing compositions containing a non-toxic adhesive polymer. While the hair treatment composition is one aspect of the present invention, a method of use in a hair extension process is also contemplated and considered to be an important aspect of the present invention.

The hair treatment composition for bonding an extension strand to a human scalp or hair proximate to a human scalp (commonly referred to as a weaving bond) preferably includes an amount of a non-toxic adhesive polymer, an amount of antioxidants and an amount of water. The amounts of the non-toxic adhesive polymer, antioxidants, and water will vary; however, the ratios must be such that the composition is still effective as an adhesive for typical hair extension processes. The amount of water may be increased or decreased depending on the desired viscosity of the final composition.

A hair treatment composition is effective if it provides at least as strong of a bond as a roughly equal amount by either weight or volume, of commonly used products such as rubber latex and isoprene butadiene.

As used herein, an extension strand is an amount of natural or synthetic hair, of any length. This strand may be many hairs combined together or as few as a single hair. The extension strand may also be an amount of natural or synthetic hair tied or sewn to a string or support material such as a plastic or cloth strip or other material as is commonly the case in the hair extension industry.

Water is defined to include de-ionized water, distilled water, purified water, filtered water, tap water or the like.

As used herein, the term “antioxidants” is defined as any compound to retard oxidation, deterioration, rancidity and gum formation. Typical antioxidants include without limitation, substituted phenolic compounds such as butylated hydroxytoluene (BHT), butylated hydroxyanisole, and propyl gallate, TOPANOL® (2,6-Di-Tert-Butyl-4-Methylphenol or 2,6-Di-Tert-Butyl-p-Cresol Butylated Hydroxytoluene) and IRGANOX® 1010 (tetrakis [methylene(3,5-di-terbutyl-4-hydroxyhydrocinnamate)]methane). Antioxidants used herein are preferably non-toxic and cause little or no skin irritation on prolonged exposure.

As used herein, the term “non-toxic” is defined as not irritating to the skin or mucous membranes of a human and not damaging to the hair or hair follicles of a human. This can be measured by examining the scalp or skin of humans who are in contact with the particular composition in question. If a particular composition does not make the scalp or skin appear to be raw, red or irritated after multiple treatments, it can be said to be non-toxic. An objective measure of non-toxicity can be found by analyzing the Material Safety Data Sheet for the particular composition. A composition which demonstrates little or no skin or eye irritation in the acute toxicity section can also be said to be non-toxic.

As used herein, the term “polymer” is defined to include both polymers, copolymers, block copolymers and mixtures thereof.

As used herein, the term “non-toxic adhesive polymer” is defined as a polymer that is both as effective as typical adhesives used in hair extension processes (such as rubber latex and isoprene butadiene), while still being non-toxic. Exemplary non-toxic adhesive polymers include without limitation, methyl vinyl ether/maleic acid, a butyl ester of the methyl vinyl ether/maleic acid polymer, an ethyl ester of the methyl vinyl ether/maleic acid polymer, an isopropyl ester of the methyl vinyl ether/maleic acid polymer. The most preferable non-toxic adhesive polymer is methyl vinyl ether/maleic acid or 2-butenedioic acid (Z)-, polymer with methoxyethene, sold commercially under the trademark Gantrez® S-97, by International Specialty Products, Inc., CAS Registry No. 00025153-40-6. The molecular formula of this polymer is (C₄H₄O₄*C₃H₆Oₙ.

In addition to the non-toxic adhesive polymer, antioxidants and water; additives such as fragrances, conditioning agents, coloring agents, preservatives, etc. may also be included in the hair treatment composition to provide additional desired properties. Exemplary additives include without limitation, siloxanes such as decamethylcyclopentasiloxane, ether siloxanes such as polysiloxane polyether copolymer, polyols such as dimethicone copolyol, organoacetates such as polydimethyl ammonium acetate copolymer, organic salts such as quatemium-80, cocoamidopropyl dimethyl glycines such as cocoamidopropyl betaine and glycols such as 1-4 tetramethylene glycol.

Preferably, the amount of the non-toxic adhesive polymer in the hair treatment composition is in the range of about 50% to about 95% by weight, more preferably in the range of about 65% to about 80% by weight and most preferably about 75% by weight of the hair treatment composition.

Preferably, the amount of antioxidants in the hair treatment composition are in the range of about 1% to about 6% by weight, more preferably in the range of about 2% to about 5% by weight and most preferably about 3% by weight of the hair treatment composition.

Preferably, the amount of water in the hair treatment composition is in the range of about 4% to about 44% by weight, more preferably in the range of about 18% to about 33% by weight, most preferably about 22% by weight of the hair treatment composition.

For purposes of this application, use of the term “about” is defined as the amount that results in an insubstantial change in the overall performance of the hair treatment composition or the amount that is attributable to experimental error in measurement of the individual components of the composition. “About” should not typically exceed 5%.

The hair treatment composition is typically prepared by mixing desired amounts of non-toxic adhesive polymer, antioxidants and water together. The amount of nontoxic adhesive polymer in the composition can be objectively determined prior to preparation of the composition by simple weight percent calculations or can be determined after preparation of the composition by simple acid/base titration using standardized sodium hydroxide as the titrant and phenolphthalein as the indicator. The reaction ratio is 1 mol of hair treatment composition per 2 mols of NaOH.

The above-described hair treatment composition is also preferably used in a method to extend the length of a customer's hair. This method preferably includes providing a human subject (or customer) who desires to increase the length of his/her hair. This method also could be used for someone who desires to increase the amount of his/her hair. This latter option is desirable for those persons whose hair is thinning with age.

The method preferably begins by obtaining an amount of extension strands, which are preferably tied or otherwise attached to a string or other support. Next, the hair on the customer's head is parted, preferably near the crown of the head, thereby exposing a portion of the customer's scalp or the hair proximate to the customer's scalp. The extension strands are then placed proximate to the scalp such that the string of the extension strand is in very close proximity to the scalp. It is preferable that the string is in direct contact with hair proximate to the scalp. An amount of the hair treatment composition is then applied to the area of the hair proximate to the scalp where the string is placed in an amount sufficient to bond the strand extension to hair proximate to the customer's scalp. The non-toxic adhesive polymer is preferably included in the hair treatment composition as described above. Preferably, the string is pressed onto the hair proximate to the scalp to facilitate the bonding of the strand extension. The hair treatment composition is then allowed to dry and the bond is thereby set. The drying time (or bonding set time) usually takes anywhere from 5 seconds to 100 seconds depending on the viscosity characteristic of the non-toxic adhesive polymer. Generally, as the viscosity of the non-toxic adhesive polymer increases, the bonding set time increases. This process is then preferably repeated by creating another part in the customer's hair next to the previous part and working away from the crown of the head (typically in both directions) until all the desired area of the customer's head is treated.

The amount of non-toxic adhesive polymer applied to hair proximate to the scalp must be sufficient to bond the strand extension to hair proximate to the customer's scalp. As a rule of thumb, the amount laid down on hair proximate to the scalp is preferably approximately 3 ml of the hair treatment composition prepared according to the Example below per 12-inch area. This area is defined as a straight line over the sides and top of the head. Of course, this amount will vary depending on the amounts of non-toxic adhesive polymer in the composition, the viscosity of the composition, desired bond strength, etc.

The hair treatment composition of the present invention achieved advantageous and unexpected results in bonding natural hair to synthetic and/or human hair with the result of lengthening the hair and fulfilled a long felt need in the industry for bonding hair extension to hair proximate to the scalp with reduced toxicity.

As used herein, the term bond is defined as a semi-permanent bond between the extension strand and hair proximate to the scalp which cannot be easily broken by manually pulling on the extension strand. Preferably, this bond is of equal or greater strength than those bonds achieved by use of typical polymers such as rubber latex or isoprene butadiene. More preferably, the bond is such that it lasts until the bond is chemically broken by treatment with a product known as a hair bond remover.

To remove the extension strands from the scalp or form hair proximate to the scalp, an amount of hair bond remover is applied to scalp or to hair proximate to the scalp. This weakens the bond and allows for the extension strand to be easily separated from the scalp or from hair proximate to the scalp. Preferably, the hair bond remover according to the present invention includes an amount of the non-toxic adhesive polymer used to initially create the bond. Additionally, the hair bond remover preferably includes oxidants such as ethanol and hydrogen peroxide, moisturizers such as aloe vera and vitamins such as vitamin E. The remaining ingredients are preferably added to facilitate removal of the remaining nontoxic adhesive polymer and for revitalizing and replenishing the customer's hair.

Preferably, a hair bond remover in accordance with the present invention includes about 1% to about 10% by weight of the non-toxic adhesive polymer, more preferably about 3% to about 6% by weight of the non-toxic adhesive polymer and most preferably about 5% by weight of the non-toxic adhesive polymer.

The amount of non-toxic adhesive polymer in the hair bond remover must be an amount sufficient to weaken or re-liquify the original bond between the extension strand and the scalp or hair proximate to the scalp. This amount can be experimentally determined by applying the hair bond remover and gently pulling on the extension strand. If the strand can be removed, this amount is considered sufficient. If the extension strand cannot be removed, an additional amount of hair bond remover is required to be applied. This step is repeated until the bond is broken and the extension strand is removed from the scalp or from hair proximate to the scalp.

Because it is preferable to remove the bond created by the hair treatment composition by applying a hair bond remover with the same non-toxic adhesive polymer, it is also preferable to combine the hair treatment composition and the hair bond remover into a system for convenient packaging and sale. This system would preferably include a container of the hair treatment composition discussed above and a container of the hair bond remover discussed above. This system would more preferably include a bulk amount of the non-toxic adhesive polymer, a bulk additive of antioxidants and a bulk additive of moisturizers, oxidants and vitamins. A hair stylist would then have the ability to mix the preferred amount of non-toxic adhesive polymer and antioxidants together with water to create a suitable hair treatment composition. This same hair stylist could then prepare a hair bond remover by mixing the preferred amount of non-toxic adhesive polymer, the preferred amount of moisturizers, oxidants and vitamins and water together. The proper amounts of each ingredient in the system can also be prepackaged for hair stylists in pre-measured packets. The hair stylists then need only to open each packet and mix the contents of the packets. This system provides hair stylists with significant flexibility in preparing and using the hair treatment compositions and hair bond removers according to the present invention. Further, it provides hair stylists with the ability to add their own additives to the hair treatment compositions and hair bond removers to suit each individual customer's needs.

Lastly, it is preferable that the customer's hair is washed with a neutralizing shampoo to neutralize the effects of the hair bond remover and bring the pH of the customer's hair back to normal. Typical hair extension and removal processes leave the customers hair in the pH range of about 3-5. This range is not healthy for normal hair. The purpose of this final step is to remove all remaining amounts of the hair treatment composition or hair bond remover remaining. Thus, treatment with a neutralizing shampoo returns the pH of the hair to normal ranges (6-8). This prevents any additional long-term damage to the hair. One such neutralizing shampoo is Joyce Williams' Neutralizing Shampoo.

### EXAMPLE

A weaving bond in accordance with the present invention was prepared as follows:

1) 8 mL of liquid GANTREZ(VS-97 (a free flowing powder of a polymer of methyl vinyl ether/maleic acid) was mixed with 1.7 mL of deionized water;

2) 0.3 mL of BHT was then mixed into the mixture of GANTREZ®S-97 (a free flowing powder of a polymer of methyl vinyl ether/maleic acid) and de-ionized water.

This particular hair treatment composition was the example used to experimentally determine the amount of non-toxic adhesive polymer sufficient to bond the strand extension to hair proximate to the scalp in accordance with the above-described method.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A hair treatment composition for bonding extension strands to hair proximate to a human scalp comprising: about 50% to about 95% by weight of a non-toxic adhesive polymer with the balance being about 5% to 50% by weight of a mixture of antioxidants and water.

2. The hair treatment composition of claim 1 wherein said non-toxic adhesive polymer is about 65% to about 80% by weight.

3. The hair treatment composition of claim 1 wherein said non-toxic adhesive polymer is about 75% by weight of said hair treatment composition.

4. The hair treatment composition of claim 1, wherein said non-toxic adhesive polymer is selected from the group consisting of: a polymer of methyl vinyl ether/maleic acid, a butyl ester of said polymer of methyl vinyl ether/maleic acid, an ethyl ester of said polymer of methyl vinyl ether/maleic acid, and an isopropyl ester of said polymer of methyl vinyl ether/maleic acid.

5. The hair treatment composition of claim 1, wherein said antioxidants are selected from the group consisting of: butylated hydroxytoluene, butylated hydroxyanisole, propyl gallate, (2,6-Di-Tert-Butyl-4-Methylphenol or 2,6-Di-Tert-Butyl-p-Cresol Butylated Hydroxytoluene) and tetrakis methylene (3,5-di-tertbutyl-4-hydroxybydroannamate) methane.

6. The hair treatment composition of claim 3 wherein said non-toxic adhesive polymer is methyl vinyl ether/maleic acid.

7. A method of extending the length of a customer's hair comprising the steps of: exposing a portion of said customer's scalp, placing an extension strand proximate to said scalp, applying an effective amount of a hair treatment composition having about 50% to about 95% by weight of a non-toxic adhesive polymer with the balance being about 5% to 50% by weight of a mixture of antioxidants and water to the hair proximate to said scalp, bonding an extension strand to said hair with hair treatment composition.

8. The method of claim 7, wherein said non-toxic adhesive polymer is selected from the group consisting of a polymer of methyl vinyl ether/maleic acid, a butyl ester of said polymer of methyl vinyl ether/maleic acid, an ethyl ester of said polymer of methyl vinyl ether/maleic acid, and an isopropyl ester of said polymer of methyl vinyl ether/maleic acid.

9. The method of claim 7, wherein said non-toxic adhesive polymer is methyl vinyl ether/maleic acid.

10. The method of claim 7, wherein said non-toxic adhesive polymer is about 65% to about 80% by weight of said hair treatment composition.

11. The hair treatment composition of claim 6, wherein said non-toxic adhesive polymer is about 75% by weight of said hair treatment composition.

12. The method of claim 7 further comprising the steps of applying an amount of hair bond remover having an amount of said non-toxic adhesive polymer in an amount sufficient to loosen said bond between said extension strand and said natural scalp hair and separating said extension strand from said hair.

13. The method of claim 12 further comprising the steps of neutralizing the hair and scalp of said customer by applying an amount of neutralizing shampoo effective to restore the natural pH of said customer's hair.

14. A hair extension and removal system comprising: a hair treatment composition to bond an extension strand to hair proximate to a scalp and a hair bond remover to remove said extension strand from said hair, said hair treatment composition having about 50% to about 95% by weight of a non-toxic adhesive polymer with the balance being about 5% to about 50% of water and antioxidants, said hair bond remover having an amount of said non-toxic adhesive polymer sufficient to loosen a bond created by applying an amount of said hair treatment composition and allow for removal of an extension strand from said hair.

15. The hair extension and removal system of claim 14 wherein said hair bond remover comprises about 1% to about 10% of said non-toxic adhesive polymer.

16. The hair extension and removal system of claim 14, wherein said hair bond remover comprises about 5% by weight of said non-toxic adhesive polymer.

17. A hair bond remover comprising: an amount of non-toxic adhesive polymer sufficient to loosen a bond between an extension strand and a customer's scalp that was created using a hair treatment composition having an amount of non-toxic adhesive polymer and further comprising a mixture of moisturizers, oxidants and vitamins.

18. The hair bond remover of claim 17 wherein said amount of non-toxic adhesive polymer is about 1% to about 10% by weight with the balance being about 90% to about 99% by weight of a mixture of moisturizers, oxidants and vitamins.

19. The hair bond remover of claim 17 wherein said amount of non-toxic adhesive polymer is about 3% to about 6% by weight with the balance being about 94% to about 97% by weight of a mixture of aloe vera, oxidants and vitamin E.

20. The hair extension and removal system of claim 14, wherein said non-toxic adhesive polymer is selected from the group consisting of a polymer of methyl vinyl ether/maleic acid, a butyl ester of said polymer of methyl vinyl ether/maleic acid, an ethyl ester of said polymer of methyl vinyl ether/maleic acid, and an isopropyl ester of said polymer of methyl vinyl ether/maleic acid.
